(19) United States
(12) Patent Application Publication
(10) Pub. No.: US 2021/0246482 A1
(43) Pub. Date: Aug. 12, 2021

(54) ENVIRONMENTAL PROFICIENCY METHODS IN A FOOD PRODUCTION ENVIRONMENT

(71) Applicant: Cherney Microbiological Services, Ltd., Green Bay, WI (US)

(72) Inventors: Stephen Michael Kuchenberg, Abrams, WI (US); Ashley Anne Erdman, Green Bay, WI (US); Katie Lynn Mattson, Oconto, WI (US)

(73) Assignee: Cherney Microbiological Services, Ltd., Green Bay, WI (US)

(21) Appl. No.: 16/787,785

(22) Filed: Feb. 11, 2020

(51) Int. Cl. C12Q 1/10; C12Q 1/14; C12Q 1/22; C12Q 1/24

(52) U.S. Cl. CPC C12Q 1/10; C12Q 1/24; C12Q 1/22; C12Q 1/14

(57) ABSTRACT

Methods for monitoring the environmental hygiene of an area includes inoculating one or more test items or sampling devices with at least one target and providing the inoculated test items or sampling devices for testing using at least one standard detection protocol. The test is reviewed to determine whether the protocol solicits a reaction to each target. If a target solicits the intended result, then the standard detection protocol for that target is determined to be proficient. Methods for monitoring the effectiveness environmental hygiene for an area to ascertain the proficiency to the testing or sampling of at least one test surface includes providing at least one target and testing or sampling the test surface. A test item or sampling device is tested with at least one standard detection protocol and the test is reviewed to determine whether the at least one target is detected as intended by the protocol.

### CROSS-REFERENCE TO RELATED APPLICATION(S)

N/A

### FIELD

The disclosure is related to the field of environmental testing; for example, the testing of food, materials, surfaces, and/or equipment, for instance surfaces or equipment with which food comes into contact during preparation, processing or storage.

### BACKGROUND

Determination of cleanliness in industrial settings is important for maintaining good hygiene and sanitation. It is estimated that there are over 33,000 facilities producing, handling, or storing food or food ingredients in the US. The surfaces of equipment used for food handling, storage or processing are major sources of microbial and allergen contamination. Microbial contamination can lead to food spoilage and, if pathogens are present, food poisoning or transmission of disease. This may lead to brand value damage and hurt profits.

One program that exists to identify where in a food service establishment the likely sources of contamination exist is referred to as HACCP (Hazard Analysis and Critical Control Points) and is a systematic preventative approach to food safety. However, the program, although diligent and structured, merely identifies the Obvious ways to control pathogens, namely employee hand washing and the rigorous separation between uncooked and cooked foods before, during and after preparation.

In order for a food manufacturer or handler to effectively comply with HACCP-based requirements or standards, it is vital that the food manufacturer or handler have an effective system in place to collect, monitor, and analyze relevant HACCP data. The necessity for this can be seen by examining the seven HACCP principles required for compliance. 1. Conduct a hazard analysis. 2 Determine the critical control points (CCP). A CCP is a point, step or procedure in a food process where a number of possible measurement controls can be applied and, as a result, a food safety hazard can be prevented, eliminated, or reduced to acceptable levels. 3. Establish measurement parameters and critical limits for each CCP and identify methods for measuring the CCP. For example, compliance with a cooking CCP may be assessed by the combination of two indicators: time and temperature. 4. Monitor the CCP to ensure ongoing compliance with established critical limits. A monitoring system should not only detect individual deviations, but also analyze data to identify patterns of deviation that could indicate a need to reassess the HACCP plan. 5. Establish corrective actions to be taken when monitoring of important parameters shows that a critical limit has not been met. 6. Maintain accurate records. Effective record keeping is a requirement. HACCP records must be created at the time events occur and include the parameter measurement, date, time and the plant employee making the entry. 7. Verify that the system is working properly initially as well as ongoing. These activities include calibration of the monitoring equipment, direct observations of the monitoring activities and a review of the records.

One essential characteristic of the HACCP system that differentiates it from previous inspection system(s) is that it places responsibility for food safety directly on the food manufacturer or handler. Each food processor or handler must be able to identify CCPs, measure a variety of parametric indicators for each CCP (e.g., time and temperature measurements to verify a cooking process), identify deviations, perform trend analysis of deviations, and document the data to show compliance with the HACCP requirements.

It is not surprising that the growing reach of HACCP-based systems is progressing concurrently with a trend toward methods of testing that are improved by being more rapid, more sensitive and easier to perform. More stringent standards, such as those associated with HACCP-based systems, are expected to motivate such improvements in methods of testing. The reverse is also true in that as test methods improve, standards are likely to become more stringent, since compliance can be more accurately, precisely, and efficiently maintained and verified.

Still, there is currently a lack of environmental proficiency programs, especially when it comes to internal environmental monitoring program effectiveness. As the testing requirements become more stringent, so too must the monitoring of the testing to ensure compliance and proficiency, particularly at CCPs. By providing appropriate proficiency programs, facilities will be able to provide evidence their practices and techniques are effective and provide a safe production environment for food and food ingredients.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

A method for monitoring the environmental hygiene of an area is disclosed. An area, such as a CCP at a food production facility, is monitored for causative agents by an environmental monitor. A test item or sampling devices, for example a swab or a plurality of swabs, are inoculated with at least one target. The targets may be causative agents, allergens, toxins, toxic chemicals or other target organisms, including but not limited to, biological pathogens that cause disease, such as viruses, parasites, fungi, or bacterium. The targets may be a specific type of pathogenic bacteria, including but not limited to, Escherichia coli, Shigella bacteria, Salmonella bacteria, Listeria bacteria, Yersinia bacteria, or Staphylococcus bacteria. The test item or sampling devices are provided to the environmental monitor for the area to be tested for proficiency. The environmental monitor tests the test item or sampling devices using at least one standard detection protocol or protocols for the area. The testing may occur within the area, adjacent the area, near the area, or remotely from the area by a proficiency provider or other testing service. The result of the test is determined and reviewed. In one embodiment, the test is reviewed to determine whether the at least one standard organism detection protocol solicits a reaction to each target. If a target is identified by the test and deemed proficient by proficiency protocols, then the standard detection protocol for that target is determined to be proficient. The standard detection protocol may include one or more discrete tests for one or more causative agents or targets.

In certain embodiments, the step of inoculating the test item or sampling device with at least one target contemplates inoculating the test item or sampling devices with a target that solicits a positive reaction to the at least one standard detection protocol. In other embodiments, a negative reaction or cross reaction is solicited. In certain embodiments test items or sampling devices such as a swab or plurality of swabs are inoculated with at least two targets: a first target that solicits a positive reaction to the at least one standard detection protocol, and a second target that solicits a negative reaction to the at least one standard detection protocol. In all embodiments, a negative reaction could also be a test item or sampling device with no target added, e.g., an uninoculated swab. In other embodiments, test items or sampling devices such as swabs are inoculated with at least three targets: a first target that solicits a positive reaction to the at least one standard detection protocol, a second target that solicits a negative reaction to the at least one standard detection protocol, and a third target that solicits one of a positive reaction, and negative reaction or a cross reaction to the at least one standard detection protocol. Again, a negative reaction could also be a solicited with a no target added, uninoculated test item or sampling device. In still other embodiments, the step of inoculating the test item or sampling device with at least one target contemplates inoculating the test item or sampling device with a target that solicits a cross reaction to at least one standard detection protocol. The step of reviewing the testing step to determine whether the at least one standard organism detection protocol solicits a reaction to the target further comprises determining whether a first at least one standard organism detection protocol positively identifies the target and whether then a second at least one standard detection protocol negatively identifies the target. Any number of test items, sampling devices and targets may be used; the number of targets that may be used is only limited by the practicalities of cross reactions, overloading, sensitivity of the standard detection protocols, and other physical or chemical limitations.

A method for monitoring the effectiveness environmental hygiene for an area is also disclosed. This method may ascertain the proficiency to the testing or sampling, e.g. swabbing, of at least one test surface of an area such as a CCP. At least one target is provided to the environmental monitor, and the at least one target is placed on the test surface. The surface is tested or sampled to obtain a test item or sampling device that is then collected for testing. The test incorporates at least one standard detection protocol that are known in the art to detect the presence of causative agents, such as pathogenic bacteria molds or yeasts. The test is reviewed to determine whether the at least one target is detected by the standard detection protocol.

The test surface may be provided by a proficiency provider. The test of the may involve sending the test item(s) or sampling device(s) such as a swab to the proficiency provider and having the proficiency provider test the test item(s) or sampling device(s) with at least one standard detection protocol. In certain embodiments, the step of placing the at least one target on a test surface is eliminated, and the step of providing at least one target to the environmental monitor contemplates pre-depositing at least one target on a test surface and providing that test surface with the pre-deposited target or targets to the environmental monitor. The test surface may be provided with at least one target as a kit. In certain instances, the kit includes a test surface with at least one pre-deposited target on the test surface.

In certain embodiments, the step of providing at least one target involves providing the at least one target organism. In certain embodiments, the at least one target is provided in a liquid, solid or a dehydrated form. In other embodiments, the at least one target is one of at least one live target organism, at least one dead target organism or at least one lyophilized target organism. The targets may be causative agents, allergens, toxins, toxic chemicals or other target organisms, including but not limited to, biological pathogens that cause disease, such as viruses, parasites, fungi, or bacterium. The live, dead or lyophilized target organism may be a specific type of pathogenic bacteria, including but not limited to one of Escherichia coli, Shigella bacteria, Salmonella bacteria, Listeria bacteria, Yersinia bacteria, Staphylococcus bacteria, or other certain bacteria, molds or yeasts. In other embodiments, the at least one target is a specific DNA primer and the testing detects for the at least one a specific DNA primer. The at least one specific DNA primer may be a specific type of pathogenic bacteria, including but not limited to, one of Escherichia coli, Shigella bacteria, Salmonella bacteria, Listeria bacteria, Yersinia bacteria, Staphylococcus bacteria, or other certain bacteria, molds or yeasts. In still other embodiments, the step of providing at least one target involves providing a target that solicits a negative reaction to at least one standard detection protocol. In all embodiments, a negative reaction could also be a test item or sampling device with no target added, e.g., an uninoculated swab. In still other embodiments, the method contemplates that the step of providing at least one target to the environmental monitor includes providing at least one a specific DNA primer as the at least one target by a proficiency provider, and the step of testing the test item or sampling device with the at least one standard detection protocol further is accomplished by an entity other than the proficiency provider

### BRIEF DESCRIPTION OF THE DRAWINGS

FIG. 1 demonstrates a method for monitoring the environmental hygiene of an area, specifically testing the proficiency of the testing at an area.

FIG. 2a demonstrates providing more than one target to a test item or sampling devices.

FIG. 2b demonstrates providing multiple targets to multiple test items or sampling devices.

FIG. 3 demonstrates a method for monitoring the environmental hygiene of an area, specifically testing the proficiency of testing or sampling techniques for an area.

### DETAILED DESCRIPTION

In the present description, certain terms have been used for brevity, clarity and understanding. No unnecessary limitations are to be inferred therefrom beyond the requirement of the prior art because such terms are used for descriptive purposes only and are intended to be broadly construed. The different methods and assemblies described herein may be used alone.

The environmental proficiency programs disclosed herein will provide evidence that food production facility, e.g. food producer, manufacturer, or food handling facilities, have practices that are proficient at collecting and/or producing appropriate results in regard to internal environmental monitoring sampling and/or testing. By participating in the program with acceptable results, the facility can show evidence of being proficient in the process. Being proficient demonstrates appropriate practices to aid in food safety. As standards and policies shift towards being proactive about food safety rather than reactive, these programs will demonstrate standard compliance.

The present application contemplates multiple methods of testing the proficiency of environmental compliance protocols. One set of methods monitors the environmental hygiene of an area by ensuring that the detection protocols used in that area detect certain targets, for example target organisms such as, but not limited to, Escherichia coli, Shigella bacteria, Salmonella bacteria, Listeria bacteria, Yersinia bacteria, Staphylococcus bacteria, or other certain bacteria, toxins, allergens, mold or yeast. A second set of methods are used to monitor the effectiveness of environmental hygiene for an area so that the effectiveness of testing or sampling techniques and protocols can be verified as proficient in detecting targets.

The term “bacteria” refers to unicellular microorganisms. The bacteria species may be eubacteria, cyanobacteria or archaebacteria. Bacteria may be prokaryotes, typically up to about one micron in length. Individual bacteria may have a wide range of shapes including spheres to rods to spirals. Bacteria may be Gram-positive or Gram-negative. Gram-positive bacteria possess a thick wall containing layers of peptidoglycan and teichoic acids. Gram-negative bacteria have a thin cell wall consisting of a few layers of peptidoglycan surrounded by a second lipid membrane containing lipopolysaccharides and lipoproteins. Some bacteria require an eukaryotic host for replication, some form spores, and some may form or participate in biofilm formation. As used herein, “toxins” refers to toxic substances produced by living cells or organisms that can cause disease in contact with or by adsorption by body tissue. As used herein “mold” refers to species of microscopic fungi that grow in the form of multicellular filaments called hyphae. In contrast, microscopic fungi that grow as single cells are called “yeasts.” Both molds, yeasts and bacteria may include spores of the same which are differentiated developmental structures that are adapted for dispersion and surviving for extended periods of time in unfavorable conditions. “Allergens” refers to purified allergens, allergenic fragments of an allergen, extracts from a source of allergen, and the like. Allergens of interest include food allergens, chemical allergens (e.g., drugs, cosmetics, and the like), plant-derived allergens, and animal-derived allergens. Allergens of interest according to the present invention include antigens found in foods such as fruits, peanuts, peanut oil, other nuts, milk proteins, egg whites, shellfish, tomatoes, etc.; airborne antigens such as grass pollens, animal danders, house mite feces, etc.; drug antigens such as penicillins and related antibiotics, sulfa drugs, barbiturates, anticonvulsants, insulin preparations (particularly from animal sources of insulin); insect venoms and agents responsible for allergic dermatitis; and latex. The specific allergen may be any type of chemical compound such as, for example, a polysaccharide, a fatty acid moiety, a protein, etc.

Environmental hygiene is accomplished through sampling of an area, such as the production environment, at critical control points (CCP). Sampling may provide evidence of how causative agents, such as pathogenic bacteria molds or yeasts, were introduced and proliferated in a food chain (farm-to-table). Sampling may also demonstrate the effectiveness of controls, e.g. standard detection protocols, and/or preventive measures. Individuals conducting a test, typically the sampler or environmental monitor, use a test item or sampling device or other a specialized tool for collecting pathogens, e.g. a swab, that is wiped over a surface and submitted for analysis. The test item(s) or sampling device(s) may be, for example, but not limited to, a hand held sponge, a sponge on a stick, a swab or a swab tube. The methods disclosed herein test the effectiveness of environmental hygiene by both determining the proficiency of controls and the proficiency of collecting the input into such controls by determining the proficiency of the swab and swabbing techniques.

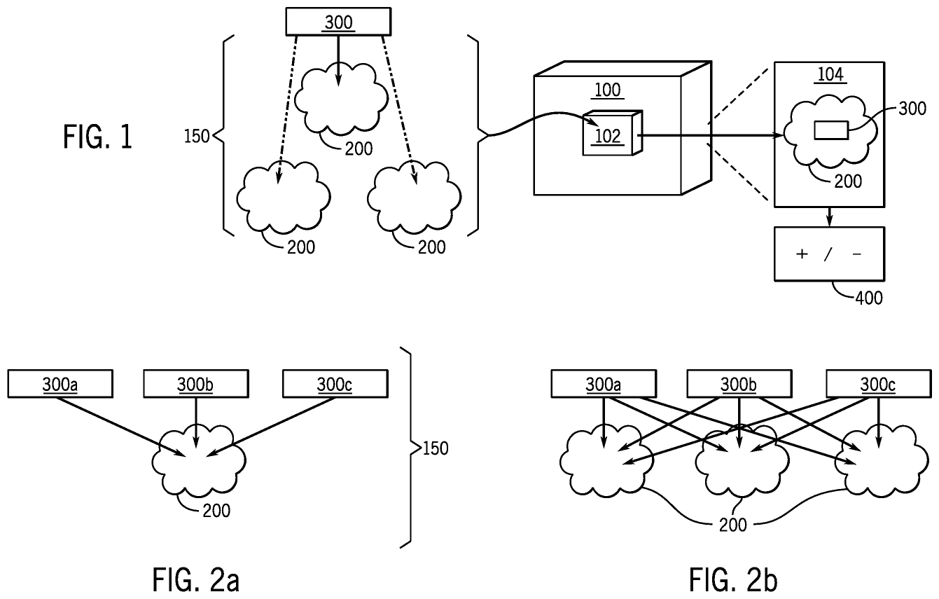

Turning now to FIG. 1, a method for monitoring the environmental hygiene of an area is demonstrated. An area 100, such as a CCP at a food production facility, is monitored for causative agents. Such monitoring may be accomplished by an environmental monitor 102. The environmental monitor 102 may be a person, an automated testing apparatus, or a combination of both. For example, the environmental monitor 102 may include a sampler that tests or samples surfaces and places a test item(s) or sampling device(s) into collection area of testing. Alternatively, the environmental monitor 102 may be an automated machine that tests or samples an area 100 and collects test item(s) or sampling device(s) for testing. The test 104 may be conducted in, at or near the area 100 or at a separate location away from the area 100. The test 104 incorporates at least one standard detection protocol that are known in the art to detect the presence of causative agents, such as pathogenic bacteria molds or yeasts.

In one embodiment, the a method for monitoring the environmental hygiene contemplates obtaining test items or sampling devices 200, for example a swab or a plurality of swabs, and inoculating 150 one or more of the test items or sampling devices 200 with a target 300. In certain embodiments, the test items or sampling devices 200 may be inoculated with one or more targets 300. In other embodiments, the test items or sampling devices 200 may be inoculated with two or more targets 300a, 300b. In still other embodiments, the test items or sampling devices 200 may be inoculated with three or more targets 300a, 300b, 300c, 300_{∞}. The targets 300 may be causative agents, toxins, toxic chemicals or other target organisms, including but not limited to, biological pathogens that cause disease, such as viruses, parasites, fungii, or bacterium. In certain embodiments, the targets 300 are a specific type of pathogenic bacteria, including but not limited to, Escherichia coli, Shigella bacteria, Salmonella bacteria, Listeria bacteria, Yersinia bacteria, or Staphylococcus bacteria. A test item or sampling device 200 is inoculated though techniques known in the art, including but not limited to introducing the test item or sampling device to cultured media of the targets 300. The inoculation of the test items or sampling devices 200 with the targets 300 may occur at the location of supplier of the inoculated test items or sampling devices 200, including a proficiency provider or may occur within the area 100, adjacent the area 100, near the area 100 or remotely from the area 100. As used herein, a “proficiency provider” is an individual or organization that provides testing and other laboratory services to determine the performance of individual laboratories or CCPs for specific tests or measurements and is used to monitor laboratories' or CCP's continuing performance by comparing the measuring results obtained by different laboratories or CCPs.

The test items or sampling devices 200 are provided to the environmental monitor 102 for the area 100 to be tested for proficiency. The environmental monitor 102 tests the test items or sampling devices 200 using at least one standard detection protocol or protocols for the area 100. As noted, the step of testing 104 may occur within the area 100, adjacent the area 100, near the area 100 or remotely from the area 100. The method contemplates a determination or review step 400 where the result of the testing step 104 are reviewed. In one embodiment, the testing step 104 is reviewed to determine whether the at least one standard organism detection protocol solicits a reaction to each target 300. If a target 300 is identified by the testing step 104, then the standard detection protocol for that target 300 is determined to be proficient. As noted, the standard detection protocol of the testing step 104 may detect for one or more of the following targets: Escherichia coli, Shigella bacteria, Salmonella bacteria, Listeria bacteria, Yersinia bacteria, Staphylococcus bacteria, toxins mold or yeast, or a combination of the same. The standard detection protocol of the testing step 104 may include one or more discrete tests for one or more causative agents or targets. In other words, the standard detection protocol of the testing step 104 may test for one, two, three or more discrete causative agents or targets.

In certain embodiments, the step of inoculating 150 the test items or sampling devices 200 with at least one target 300 contemplates inoculating 150 the test items or sampling devices 200 with a target 300 that solicits a positive reaction to the at least one standard detection protocol of the testing step 104. In other words, the target 300 is designed to positively identify the presence of a causative agent. In this embodiment, the step of reviewing 400 the testing step 104 to determine whether the at least one standard organism detection protocol solicits a reaction to the target 300 contemplates determining whether the at least one standard organism detection protocol positively identifies the target 300. In other embodiments, the step of inoculating 150 the test items or sampling devices 200 with at least one target 300 contemplates inoculating 150 the test items or sampling devices 200 with a target 300 that solicits a negative reaction to the at least one standard detection protocol of the testing step 104. In other words, the target 300 is designed to identify the absence of a causative agent. In this embodiment, the step of reviewing 400 the testing step 104 to determine whether the at least one standard organism detection protocol solicits a reaction to the target 300 contemplates determining whether the at least one standard organism detection protocol negatively identifies the target 300. In all embodiments, a negative reaction could also be a test item or sampling device 200 with no target added, e.g., an uninoculated swab. In this instance, the step of reviewing 400 the testing step 104 to determine whether the at least one standard organism detection protocol solicits a reaction to the target 300 still contemplates determining whether the at least one standard organism detection protocol negatively identifies the target 300. In still other embodiments, the step of inoculating 150 test items or sampling devices 200 with at least one target 300 contemplates inoculating 150 the test items or sampling devices 200 with a target 300 that solicits a cross reaction to the at least one standard detection protocol of the testing step 104. In other words, the target 300 is designed to solicit a reaction that should be negative, but sometimes elicits a positive result, indicating that additional testing should be performed. In this embodiment, the step of reviewing 400 the testing step 104 to determine whether the at least one standard organism detection protocol solicits a reaction to the target 300 contemplates determining whether the at least one standard organism detection protocol positively identifies the target 300, which is later determined to be negative using follow up testing.

As shown in FIGS. 2a and 2b, in certain embodiments test items or sampling devices 200 are inoculated 150 with at least two targets 300a, 300b. The test items or sampling devices 200 may be inoculated 150 with a first target 300a that solicits a positive reaction to the at least one standard detection protocol, and a second target 300b that solicits a negative reaction to the at least one standard detection protocol. In other embodiments, the test items or sampling devices 200 are inoculated 150 with at least three targets 300a, 300b, 300c, The test items or sampling devices 200 may be inoculated 150 with a first target 300a that solicits a positive reaction to the at least one standard detection protocol, a second target 300b that solicits a negative reaction to the at least one standard detection protocol, and a third target 300c that solicits one of a positive reaction, and negative reaction or a cross reaction to the at least one standard detection protocol. It must be noted that any number of targets 300 and test items or sampling devices 200 may be used, and that the number of targets 300 and test items or sampling devices 200 that may be used in this protocol is only limited by the practicalities of cross reactions, overloading, sensitivity of the standard detection protocols, and other physical or chemical limitations.

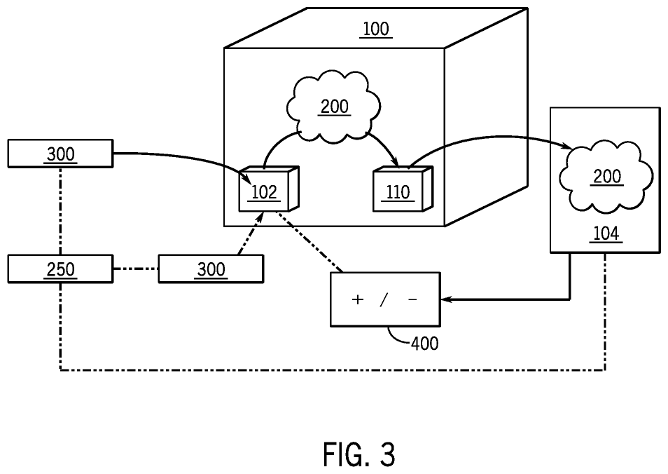

Turning now to FIG. 3, a method for monitoring the effectiveness of environmental hygiene for an area is demonstrated. The method is applied to an area 100 having an environmental monitor 102 to ascertain the proficiency to the testing or sampling of at least one test surface 110 in the area 100. The method contemplates providing at least one target 300 to the environmental monitor 102. The at least one target 300 is placed on the test surface 110 in the area 100, and the surface 110 is tested or sampled to obtain at least one test item or sampling device 200. The test items or sampling devices 200 are then collected for testing. The test 104 may be conducted in, at or near the area 100 or at a separate location away from the area 100. The test 104 incorporates at least one standard detection protocol that are known in the art to detect the presence of causative agents, such as pathogenic bacteria molds or yeasts. The test 104 is reviewed to determine 400 whether the at least one target 300 is detected by the standard detection protocol.

The test surface 110 may be provided by a proficiency provider 250. The test 104 of the test item(s) or sampling device(s) 200 with the at least one standard detection protocol may involve sending the test item(s) or sampling device(s) 200 to the proficiency provider 250 and testing the test item(s) or sampling device(s) 200 with the at least one standard detection protocol by the proficiency provider 250. In certain embodiments, the step of placing the at least one target 300 on a test surface 110 is eliminated, and the step of providing at least one target 300 to the environmental monitor 102 comprises pre-depositing at least one target on a test surface 110, and providing the test surface 110 with the pre-deposited at least one target 300 to the environmental monitor 102. Again, providing the test surface 110, with or without at least one pre-deposited target 300, may be done by the proficiency provider 250. In certain instances, the test surface 110 could be a small ceramic or plastic tile or plate, but may be any surface that mimics surface found in areas 100 having CCPs. In other instances, the test surface is provided with at least one target as a kit. In certain instances, the kit includes a test surface 110 with at least one pre-deposited target 300 on the test surface 110.

In certain embodiments, the step of providing at least one target 300 to the environmental monitor 102 involves providing the at least one target organism 300 to the environmental monitor 102. In certain embodiments, the at least one target 300 is provided in a liquid, solid or a dehydrated form. In other embodiments, the at least one target 300 is one of at least one live target organism, at least one dead target organism or at least one lyophilized target organism. Such live, dead or lyophilized target organism may be one of Escherichia coli, Shigella bacteria, Salmonella bacteria, Listeria bacteria, Yersinia bacteria, Staphylococcus bacteria, mold or yeast. In other embodiments, the at least one target 300 is a specific DNA primer and the testing step 104 detects for the at least one a specific DNA primer. This step may be done, but is not limited by being done, by the proficiency provider 250, as the standard detection protocols at the area 100, typically do not detect for DNA primers, but instead for live or dead organisms. Testing may also occur within the area 100, adjacent the area 100, near the area 100 or remotely from the area 100. Testing may be done by the proficiency provider or by an entity other than the proficiency provider. For example, the proficiency provider may partner with an separate environmental monitoring entity and create a test kit that will detect the at least one DNA primer, and the separate environmental monitoring entity, e.g. the customer of the proficiency provider, uses the test kit for testing. The at least one specific DNA primer may be specific to one of Escherichia coli, Shigella bacteria, Salmonella bacteria, Listeria bacteria, Yersinia bacteria, Staphylococcus bacteria, allergens mold or yeast.

In the above description, certain terms have been used for brevity, clarity, and understanding. No unnecessary limitations are to be inferred therefrom beyond the requirement of the prior art because such terms are used for descriptive purposes and are intended to be broadly construed. The different systems and method steps described herein may be used alone or in combination with other systems and methods. It is to be expected that various equivalents, alternatives and modifications are possible within the scope of the appended claims.

What is claimed is:

1. A method for monitoring the environmental hygiene of an area, the area having an environmental monitor and at least one standard detection protocol, the method comprising:
obtaining a test item or sampling device;
inoculating the test item or sampling device with at least one target;
providing the test item or sampling device to an environmental monitor for the area;
testing the test item or sampling device by the environmental monitor with the standard detection protocol; and
reviewing the testing step to determine whether the at least one standard organism detection protocol solicits a reaction to the target.

2. The method of claim 1, wherein the area is a critical control point for a food production, food processing or food storage facility.

3. The method of claim 1, wherein the at least one standard detection protocol detects for one or more of the Mowing: Escherichia coli, Shigella bacteria, Salmonella bacteria, Listeria bacteria, Yersinia bacteria, Staphylococcus bacteria, or other certain bacteria allergens, mold or yeast.

4. The method of claim 1 wherein the step of inoculating the test item or sampling device with at least one target comprises inoculating the test item or sampling device with a target that solicits a positive reaction to the at least one standard detection protocol and wherein the step of reviewing the testing step to determine whether the at least one standard organism detection protocol solicits a reaction to the target further comprises determining whether the at least one standard organism detection protocol positively identifies the target.

5. The method of claim 1 wherein the step of inoculating the test item or sampling device with at least one target comprises inoculating the test item or sampling device with a target that solicits a negative reaction to the at least one standard detection protocol and wherein the step of reviewing the testing step to determine whether the at least one standard organism detection protocol solicits a reaction to the target further comprises determining whether the at least one standard organism detection protocol negatively identifies the target.

6. The method of claim 1 wherein the step of inoculating the test item or sampling device with at least one target comprises not inoculating the test item or sampling device with a target such that the test item or sampling device solicits a negative reaction to the at least one standard detection protocol and wherein the step of reviewing the testing step to determine whether the at least one standard organism detection protocol solicits a reaction to the target further comprises determining whether the at least one standard organism detection protocol negatively identifies the target.

7. The method of claim 1 wherein the step of inoculating the test item or sampling device with at least one target comprises inoculating the test item or sampling device with a target that solicits a cross reaction to the at least one standard detection protocol and wherein the step of reviewing the testing step to determine whether the at least one standard organism detection protocol solicits a reaction to the target further comprises determining whether a first at least one standard organism detection protocol positively identifies the target and whether a second at least one standard detection protocol negatively identifies the target.

8. The method of claim 1 wherein the step of inoculating the test item or sampling device with at least one target comprises inoculating the test item or sampling device with two or more targets.

9. The method of claim 8 wherein the step of inoculating the test item or sampling device with at least two targets comprises inoculating the test item or sampling device with multiple targets that solicit a positive reaction, a negative reaction, or a cross reaction to the at least one standard detection protocol.

10. A method for monitoring the effectiveness of environmental hygiene for an area, the area having an environmental monitor, the method comprising:
providing at least one target to the environmental monitor;
placing the at least one target on a test surface;
testing or sampling the test surface to obtain a test item or sampling device;
testing the test item or sampling device with the at least one standard detection protocol; and
reviewing the testing step to determine whether the at east one target is detected.

11. The method of claim 10, wherein the step of placing the at least one target on a test surface is eliminated and the step of providing at least one target to the environmental monitor comprises pre-depositing at least one target on a surface, and providing the surface with the pre-deposited at least one target to the environmental monitor.

12. The method of claim 10, wherein the at least one target is a target organism and the step of providing at least one target to the environmental monitor comprises providing the at least one target in a liquid, solid or a dehydrated form.

13. The method of claim 10, wherein the step of providing at least one target to the environmental monitor further includes providing a test surface to the environmental monitor and the step of placing the at least one target on a test surface comprises placing the at least one target on the provided test surface.

14. The method of claim 10, Wherein the step of providing at least one target to the environmental monitor further comprises providing the at least one target to the environmental monitor by a proficiency provider, and the step of testing the test item or sampling device with the at least one standard detection protocol further comprises the steps of sending the test item or sampling device to the proficiency provider and testing the test item or sampling device with the at least one standard detection protocol by the proficiency provider.

15. The method of claim 10 wherein the step of providing at least one target to the environmental monitor further comprises providing at least one of at least one live target organism, at least one dead target organism or at least one lyophilized target organism.

16. The method of claim 15, wherein the step of providing at least one target to the environmental monitor comprises providing the at least one target in a liquid, solid or a dehydrated form.

17. The method of claim 15 wherein the least one target is one of Escherichia coli, Shigella bacteria, Salmonella bacteria, Listeria bacteria, Yersinia bacteria, Staphylococcus bacteria, or other certain bacteria, allergens, mold or yeast.

18. The method of claim 14 wherein the step of providing at least one target to the environmental monitor further comprises providing at least one a specific DNA primer as the at least one target, and the step of testing the test item or sampling device with the at least one standard detection protocol by the proficiency provider further comprises detecting the at least one a specific DNA primer.

19. The method of claim 10 wherein the step of providing at least one target to the environmental monitor further comprises providing at least one a specific DNA primer as the at least one target by a proficiency provider, and the step of testing the test item or sampling device with the at least one standard detection protocol further comprises detecting the at least one a specific DNA primer by an entity other than the proficiency provider.

20. The method of claim 18 wherein the least one a specific DNA primer is specific to one of Escherichia coli, Shigella bacteria, Salmonella bacteria, Listeria bacteria, Yersinia bacteria, Staphylococcus bacteria, or other certain bacteria allergens, mold or yeast.
